(19) United States
(12) Patent Application Publication
(10) Pub. No.: US 2022/0387416 A1
(43) Pub. Date: Dec. 8, 2022

(54) ATROPINE-SCOPOLAMINE WITH ENHANCED STABILITY

(71) Applicant: CMC Pharmaceuticals, Inc., Cleveland, OH (US)

(72) Inventors: Michael Lloyd RADOMSKY, Chagrin Falls, OH (US); Donald DUWE, Strongsville, OH (US)

(21) Appl. No.: 17/755,055

(22) PCT Filed: Oct. 26, 2020
(86) PCT No.: PCT/US2020/057371

§ 371 Date: Apr. 20, 2022

Related U.S. Application Data
(60) Provisional application No. 62/926,410, filed on Oct. 25, 2019.

(51) Int. Cl. A61K 31/46; A61K 47/10; A61K 31/55; A61P 25/00

(52) U.S. Cl. CPC A61K 31/46; A61K 47/10; A61K 31/55; A61P 25/00

(57) ABSTRACT

This disclosure relates to stable formulations of atropine and scopolamine for use as a medical countermeasure to combat organophosphate nerve agent threats. The formulations exploit complementary pharmacological profiles for optimal receptor blockade and anticholinergic activity within the peripheral and central nervous system. The formulations are suitable for intramuscular injection, and have stability that exceeds two years in stressed conditions.

### CROSS-REFERENCE TO RELATED APPLICATIONS

The present application claims priority to U.S. Provisional Patent Application No. 62/926,410, which was filed on Oct. 25, 2019.

### STATEMENT OF GOVERNMENT SUPPORT

This invention was made with governmental support under Contract No. W911QY18C0198 awarded by the Department of Defense. The government has certain rights in the invention.

### FIELD OF THE INVENTION

The invention relates to the field of post-exposure treatment of organophosphate intoxication.

### BACKGROUND OF THE INVENTION

Organophosphate nerve agents (OPNA) are chemical warfare agents that induce both cholinergic and non-cholinergic crisis. OPNAs include tabun, sarin, and V series, among others. Exposure to OPNAs results in accumulation of neurotransmitters at neuromuscular junctions and synapses, giving rise to neuronal overexcitation. This overexcitation may lead to altered mental status, autonomic instability, copious respiratory and oral secretions, diarrhea, vomiting, sweating, and systemic weakness and discoordination. OPNAs are harmful as liquids, vapors, or as solid particles, and can cause death within minutes of excess exposure. Consequently, OPNAs pose a serious threat to national security and military personnel.

Current countermeasures for OPNA exposure is combinatorial, commonly consisting of one or more oximes that reactive acetylcholinesterase (AChE), enabling the breakdown of neurotransmitters and assuaging symptoms of OPNA exposure. Other established treatments for OPNA exposure include anticholinergics, e.g., atropine. Atropine inhibits the overstimulation of effector cells. However, atropine alone does not adequately address nicotinic-based effects elicited by OPNAs, such as seizures and behavioral deficits, and the high doses atropine necessary for atropine to act as an effective countermeasure can be toxic.

There is currently much interest in developing and improving medical countermeasures to organophosphate nerve agents and treatment systems. Current countermeasures suffer from shelf-life instability and attendant reduced usefulness. Thus, there remains a need for organophosphate nerve agent countermeasures with improved efficacy and improved shelf-stability.

### SUMMARY OF THE INVENTION

The present disclosure relates to formulations and methods for the treatment of organophosphate nerve agent exposure.

For example, a composition to combat organophosphate nerve agent threats comprising atropine, scopolamine, and ethanol is provided, wherein the composition comprises less than 0.14% by weight of degradants twenty-four (24) or more months post synthesis.

Also disclosed is a method comprising providing a subject exhibiting a cholinergic crisis subsequent to an organophosphate compound exposure, providing a composition comprising atropine, scopolamine and ethanol that is twenty-four (24) or more months removed from synthesis, and administering said composition to said subject under conditions such that said cholinergic crisis is reduced.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one of ordinary skill in the art and having the benefit of this disclosure.

FIG. 1 presents rates of total related substance formation at 40° C. of the presently disclosed formulation and selected comparator products.

FIG. 2 presents rates of total related substance formation at 25° C. of presently disclosed formulations and selected comparator products.

FIG. 3 shows Scopolamine stability at 25° C./60% RH; 0.8 and 2.0 mg/mL, pH 3, 1 mM Citrate; Total impurities (%).

FIG. 4 shows Scopolamine stability at 25° C./60% RH; 0.8 and 2.0 mg/mL, pH 3, 1 mM Citrate; Tropic Acid levels (%).

FIG. 5 shows Scopolamine Stability at 40° C./75% RH; 0.8 and 2.0 mg/mL, pH 3, 1 mM Citrate; Total impurities (%).

FIG. 6 shows Scopolamine Stability at 40° C./75% RH; 0.8 and 2.0 mg/mL, pH 3, 1 mM Citrate; Tropic Acid levels (%).

FIG. 7 shows Impurities in Formulation C2; pH 3.25, 1 mM Citrate; Total impurities (%).

FIG. 8 shows Impurities in Formulation C2; pH 3.25, 1 mM Citrate; Tropic Acid levels (%).

FIG. 9 shows Impurities in 100% EtOH Formulation; Total impurities (%).

FIG. 10 shows Impurities in 100% EtOH Formulation; Tropic Acid levels (%).

FIG. 11 shows Formulation pH Effect; Total RS Deg Rate (%/year).

FIG. 12 shows Formulation pH Effect; Tropic Acid Deg Rate (%/year).

FIG. 13 shows Ethanol Concentration Effect; Total RS Deg Rate (%/year).

FIG. 14 shows Ethanol Concentration Effect; Tropic Acid Deg Rate (%/year).

FIG. 15 shows Regression Analysis, 25° C./60% RH; Formulation=C1 Combo: 1 mM citrate, pH 3.6; Total RS (%).

FIG. 16 shows Regression Analysis, 25° C./60% RH; Formulation=C1 Combo: 1 mM citrate, pH 3.7; Tropic Acid (%).

FIG. 17 shows Regression Analysis, 25° C./60% RH; Formulation=Combo in 100% ethanol; Total RS (%).

FIG. 18 shows Regression Analysis, 25° C./60% RH; Formulation=Combo in 100% ethanol; Tropic Acid (%).

FIG. 19 shows Regression Analysis, 25° C./60% RH; Formulation=Scopolamine only: 1 mM citrate, pH 3.8; Total RS (%).

FIG. 20 shows Regression Analysis, 25° C./60% RH; Formulation=Scopolamine only: 1 mM citrate, pH 3.9; Tropic Acid (%).

FIG. 21 shows Impurities in Formulation C1; pH 3.0, 1 mM Citrate; Total impurities (%).

FIG. 22 shows Impurities in Formulation C1; pH 3.0, 1 mM Citrate; Tropic Acid levels (%).

FIG. 23 shows Impurities in Formulation C3; pH 3.5, 1 mM Citrate; Total impurities (%).

FIG. 24 shows Impurities in Formulation C3; pH 3.5, 1 mM Citrate; Tropic Acid levels (%).

FIG. 25 shows Impurities in Formulation C4; pH 3.25, 1 mM Acetate; Total impurities (%).

FIG. 26 shows Impurities in Formulation C4; pH 3.25, 1 mM Acetate; Tropic Acid levels (%).

FIG. 27 shows Impurities in Formulation C5; pH 3.25, 1 mM Tartrate; Total impurities (%).

FIG. 28 shows Impurities in Formulation C5; pH 3.25, 1 mM Tartrate; Tropic Acid levels (%).

FIG. 29 shows Impurities in 70% EtOH Formulation; Total impurities (%).

FIG. 30 shows Impurities in 70% EtOH Formulation; Tropic Acid levels (%).

FIG. 31 shows Impurities in 50% EtOH Formulations; Total impurities (%).

FIG. 32 shows Impurities in 50% EtOH Formulations; Tropic Acid levels (%).

FIG. 33 shows Impurities in 30% EtOH Formulations; Total impurities (%).

FIG. 34 shows Impurities in 30% EtOH Formulations; Tropic Acid levels (%).

### DETAILED DESCRIPTION

The present disclosure relates to stable formulations of atropine and scopolamine for use as a medical countermeasure to organophosphate nerve agent (OPNA) exposure. The formulations exploit complementary pharmacological profiles for optimal receptor blockade and anticholinergic activity within the peripheral and central nervous system. The formulations are suitable for intramuscular injection, and their shelf life is twenty-four (24) months or more when stored at room temperature, e.g., 25° C. or from about 15° C. to about 25° C.

Presently disclosed formulations have the following advantages.

The presently disclosed formulations increase the therapeutic efficacy by exploiting atropine and scopolamine's complementary pharmacological profiles for optimal muscarinic receptor blockade and anticholinergic activity within the peripheral and central nervous systems.

The presently disclosed formulations may be provided ready to use. In exemplary embodiments, no further manipulations are needed. Examples of manipulations not required include reconstitution of a lyophilized product, or dilution into an IV bag before intramuscular (IM) administration.

The presently disclosed formulations have high bioavailability upon IM injection, and thus permit a rapid response to OPNA exposure. The formulation is provided as a sterile solution, and dissolution of suspended particles are not necessary.

The presently disclosed formulations have a shelf-life of twenty-four (24) months or more when stored at room temperature. Some formulations may have a shelf life of five years or more.

The presently disclosed formulations utilize biocompatible solvents, e.g., water, and components commonly found in FDA approved parenteral products, such as ethanol. which imparts improved stability to the formulation, sodium citrate buffer, sodium chloride and hydrochloric acid for purposes of pH adjustment.

The presently disclosed formulations do not require specialized instrumentation or manufacturing capabilities such as lyophilizers, mills, microionizers, spray dryers, specialized solid particle equipment, specialized suspension filling equipment, and the like. Further, sterile formulations suitable for injection may be formed without the need for expensive and/or highly specialized methods of insuring sterility, such as e-beam or gamma irradiation. Formulations presently disclosed are compatible with established sterile solution manufacturing methods that are well suited for a number of container closure systems including vials, syringes, and cartridges appropriate for use in auto-injectors.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

As used herein, the term “about” in the context of any of any assay measurements refers to +/−5% of a given measurement.

As used herein, the term “organophosphate compounds” refers to any compounds having structural features comprising a terminal oxygen connected to phosphorus by a double bond, i.e., a phosphoryl group; two lipophilic groups bonded to the phosphorus; and a leaving group bonded to the phosphorus, often a halide. For example, such organophosphate compounds are nerve agents including, but are not limited to, tabun, sarin, soman (GD), cyclosarin (GF), N,N-diethyl-2-(methyl-(2-methylpropoxy) phosphoryl)sulfanylethanamin (VR), and/or O-ethyl S-[2-(diisopropylamino) ethyl] methylphosphonothioate (VX). Alternatively, such organophosphate compounds are pesticides including, but not limited to, diisopropyl-fluorophosphate, azinphos-methyl, chlorpyrifos, diazinon, dichlorvos, dimethoate, ethephon, malathion, methamidophos, naled, and/or oxydemeton-methyl.

As used herein, the phrase “limit of quantitation” or “LOQ” refers to the lowest concentration at which an analyte can be reliably detected accounting for predefined bias and imprecision.

As used herein, the term “symptom” refers to any subjective or objective evidence of disease or physical disturbance observed by a subject. Evidence may include, but is not limited to, pain, headache, visual disturbances, nausea, vomiting, body temperature, complete blood count, lipid panels, thyroid panels, blood pressure, heart rate, electrocardiogram, tissue and/or body imaging scans.

As used herein, the term “accelerated stability” or “accelerated stability conditions” refers to storing a formulation or product at elevated stress conditions from normal storage condition, such as elevated temperature, e.g., 40° C.

As used herein, the term “administered” or “administering” refers to any method of providing a composition or formulation to a subject such that the composition has its intended effect on the patient. Methods of administration of formulations disclosed herein may include intramuscular autoinjectors, conventional needle and syringe, syringe assist devices, and other delivery methods known in the art such as parenteral intravenous, subcutaneous, and oral.

As used herein, the term “relative humidity” or “RH” is the ratio of the partial pressure of water vapor to the equilibrium vapor pressure of water at a given temperature.

Returning to the invention disclosure, there exists a current and future threat of chemical and biological warfare to the warfighter. Organophosphates that pose an ongoing threat are potent nerve agents, functioning by inhibiting the action of acetylcholinesterase (AChE) in nerve cells. Organophosphates can be absorbed by all routes, including inhalation, ingestion, and dermal absorption. Their inhibitory effect on the acetylcholinesterase enzyme leads to a pathologic excess of acetylcholine in the body. Their toxicity is not limited to the acute phase, however, and chronic effects have long been noted. The affected neurotransmitter acetylcholine is profoundly important in the brain's development, and many organophosphates thus have neurotoxic effects on developing organisms, even from low-levels of exposure. Other organophosphates are not toxic, yet their main metabolites, such as oxons, are.

Repeated or prolonged exposure to organophosphates may result in the same effects as acute exposure, including delayed symptoms. Other effects reported in cases of repeated exposure include impaired memory and concentration, disorientation, severe depressions, irritability, confusion, headache, speech difficulties, delayed reaction times, nightmares, sleepwalking and drowsiness or insomnia. An influenza-like condition with headache, nausea, weakness, loss of appetite, and malaise has also been reported. Even at relatively low levels, organophosphates may be hazardous to human health. The military as well as civilians are at risk, and organophosphates have been hypothesized to act on a set of brain chemicals closely related to those involved in ADHD, thus fetuses and young children, where brain development depends on a strict sequence of biological events, may be most at risk. Jurewicz et al., “Prenatal and Childhood Exposure to Pesticides and Neurobehavioral Development: Review of Epidemiological Studies” INTERNATIONAL JOURNAL OF OCCUPATIONAL MEDICINE AND ENVIRONMENTAL HEALTH (Versita, Warsaw) 21 (2):121-132 (2008).

Organophosphate poisoning, the effects of which are reported above, is one of the most common causes of poisoning worldwide. There are around 1 million cases of organophosphate poisonings per year, with several hundred thousand resulting in fatalities annually. Pandit et al., “A case of organophosphate poisoning presenting with seizure and unavailable history of parenteral suicide attempt” J EMERY TRAUMA SHOCK 4 (1):132-134 (2011); and Yurumez et al., “Acute organophosphate poisoning in university hospital emergency room patients” Intern Med 46 (13): 965-969 (2007).

As eluded to above, organophosphates inhibit AChE, causing OP intoxication by phosphorylating a serine hydroxyl residue on AChE, which inactivates AChE. AChE plays a role in nerve function, so the irreversible blockage of this enzyme, which causes acetylcholine accumulation, results in muscle overstimulation. This causes disturbances across cholinergic synapses which can only be reactivated very slowly, if at all.

Symptoms of a cholinergic crisis brought about by AChE inhibition include, but are not limited to, miosis, sweating, lacrimation, gastrointestinal symptoms, respiratory difficulties, dyspnea, bradycardia, cyanosis, vomiting, diarrhea, as well as other symptoms described above. Along with these central cholinergic effects, seizures, convulsions, coma, and/or respiratory failure are occasionally observed.

The current Department of Defense standard treatment for OPNA exposure is immediate administration of medications by autoinjector. However, current methods suffer from decreased efficacy and shelf-instability.

Charged quaternary pyridinium oximes such as pralidoxime (2-PAM) are currently used as antidotes for OPNA exposure. The major limitation of these drugs is poor CNS bioavailability owing to the drug's positive charge and lack of suitable active transporters at the blood brain barrier. Therefore, currently fielded oximes (e.g., 2-PAM, obidoxime, and HI-6) cannot directly reactivate nerve agent-inhibited AChE in the brain, which a critical target organ for OPNAs. As a result, there is little neurological protection from 2-PAM treatment.

Other antidotes for OPNA exposure may consist of a pretreatment with carbamates to protect AChE from inhibition by organophosphate compounds, and post-exposure treatments with anti-cholinergics and pyridinium oximes. Anti-cholinergic drugs work to counteract the effects of excess acetylcholine and reactivate AChE. Currently, atropine, a muscarinic receptor antagonist shown in Structure 1, is used to treat OPNA exposure. Atropine sulfate in an autoinjector, alone or in combination with oximes (pralidoxime or other pyridinium oximes such as trimedoxime or obidoxime), has been shown in non-clinical studies to treat OPNA poisoning by reactivating AChE which has undergone covalent modification by OPNAs. However, atropine cannot effectively mitigate the central muscarinic or nicotinic effects of OPNA exposure due to its limited ability to bypass the blood brain barrier. Scopolamine, a muscarinic receptor antagonist shown as Structure 2, below, is capable of permeating the blood brain behavior and is a known motion sickness preventative. The compounds disclosed herein, including scopolamine, can be obtained commercially or can be readily synthesized using techniques generally known to those of skill in the art.

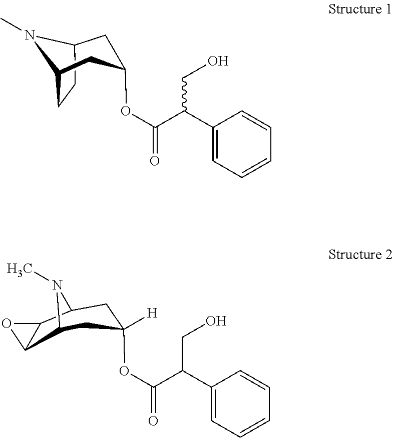

The present disclosure contemplates compositions or formulations and methods for treating and/or reversing organophosphate intoxication and symptoms of the same in a mammal attributable to exposure to agents that incapacitate the central nervous system, namely, organophosphate nerve agents. Exemplary embodiments comprise a stable combination formulation of atropine and scopolamine to be used in an autoinjector.

Herein disclosed is a formulation containing a combination product of atropine and scopolamine as a treatment against OPNAs. The combination product exploits complementary pharmacological profiles for optimal muscarinic receptor blockade and anticholinergic activity within the peripheral and central nervous system. The formulation also reduces the number of countermeasures required, as scopolamine resolves seizures and behavioral deficits associated with OP poisoning. The formulations minimize injection site necrosis and irritation, and exhibit appropriate pharmacokinetics. Formulations may optionally have a nitrogen overly, and include aqueous or non-aqueous solvents, and active or excipient concentrations. To be useful in the field, formulations are manufactured at target concentrations, filled into appropriate pharmaceutical container closures such as vials, cartridges, or syringes, and then protected from light. In the present disclosure, vials were stored at appropriate ICH (International Council for Harmonisation) conditions and other stressed conditions (e.g., 25° C./60% RH, 40° C./75% RH, and 80° C.) and then analyzed using the method discussed herein and other USP methods (i.e., pH).

Presently disclosed are aqueous formulations with stability characteristics that support a shelf life of at least 2 years. The combination aqueous solution formulations presently disclosed are composed or comprised of components commonly found in FDA-approved parenteral products (e.g., water for injection, sodium citrate buffer, sodium chloride, and pH adjusted with hydrochloric acid). Because the presently disclosed aqueous pharmaceutical formulations utilize the most biocompatible solvent (i.e., water), which is commonly utilized in many pharmaceutical unit operations and formulations, they can be injected intramuscularly at the optimum concentrations without further manipulations (i.e., reconstituted or diluted into an IV bag), and they can be systemically absorbed immediately as discussed in Savjani K T, et al., Drug Solubility: Importance and Enhancement Techniques, ISRN PHARM [Internet]. 2012 [cited 2019 Apr. 7]; 2012. Available from: https://www.ncbi.nlm.nih.gov/pmc/articles/PMC3399483/. In contrast, for example, lyophilized formulations must be reconstituted in a sterile solution, and particles in suspension must dissolve before providing effective systemic or peripheral treatment, prolonging the time between OPNA exposure and treatment, when a rapid response is paramount. See Inspection Guides>Lyophilization of Parenteral (7/93) [Internet]. [cited 2019 Apr. 7]. Available from: https://www.fda.gov/ICECl/Inspections/InspectionGuides/ucm074909.htm. This permits the manufacture of injectable solution formulations with significant large-scale capacity that requires only common compounding and aseptic filling equipment trains. No further specialized equipment is needed for manufacturing (e.g., lyophilizer, milling, micronizing, spray dryer, or specialized solid particle or suspension filling equipment) or to provide drug product sterility assurance (e.g., e-beam or gamma irradiation is not needed). Sterile solution manufacturing is well suited for a number of container closure systems that include vials, syringes, and cartridges appropriate for use in auto-injectors.

Formulations

In summary, drug product formulations containing 2 mg/mL atropine and combination (2 mg/mL atropine/3 mg/mL scopolamine) were formulated at target pH values of 2.5, 3.0, 3.25, and 3.5 in 1 mM citrate buffer. Each formulation contained 150 mM of sodium chloride (0.9%) to adjust the osmolality to approximately isotonic (e.g. 270 to 310 mOsm). The pH of the final formulation was adjusted appropriately with dilute HCl and dilute sulfuric acid (for formulations pH 2.5 and pH 3.5 as designated). Each formulation was filled in 2 mL amber borosilicate glass vials and stored at 80° C. The stability of the formulations at this stressed condition was evaluated over a three week period for appearance, assay, related substances, and pH. Extra vials were placed at room temperature conditions (25° C./60%±5% relative humidity) and accelerated stress conditions (40° C./75%±5% relative humidity).

Formulations with low levels of tropic acid and total related substances, identified as degradants, after a three month prototype stability study at 25° C., 40° C., and 80° C. are given below.

CMC Aqueous Lead (C2) Combination: 2 mg/mL atropine, 3 mg/mL scopolamine, 1 mM citrate, pH 3.25, 150 mM NaCl.

CMC Aqueous Backup (C4/C5) Combination: 2 mg/mL atropine, 3 mg/mL scopolamine, 1 mM acetate or tartrate, pH 3.25, 150 mM NaCl

CMC Non-Aqueous Lead Combination: 2 mg/mL atropine, 3 mg/mL scopolamine, ethanol (anhydrous).

CMC Non-Aqueous Backup Combination: 2 mg/mL atropine, 3 mg/mL scopolamine, propylene glycol.

The degradation rates of the above formulations are lower than comparator drug products, including commercially available monoformualtions (e.g., scopolamine only or atropine only) available from suppliers, and unbuffered aqueous formulations. The identified formulations have approximately 2 to 10 times lower degradation rates than comparative products.

Formulation Preparation and Analysis

Formulations were prepared and stored in long-term (25° C./60% RH) and accelerated storage conditions (40° C./75% RH), and evaluated at appropriate time points. Formulations were subjected to forced degradation with acid, base, heat, light and an oxidizing agent (e.g., hydrogen peroxide). In some cases, heat was combined with another agent to obtain measurable degradation.

Degraded samples were analyzed using liquid chromatography with a photodiode array detector to evaluate the purity of scopolamine and atropine peaks and each time point with a chiral purity method and an assay and related substances method.

The analysis of the formulations showed that the presently disclosed formulations have a shelf life of on or about 60 months; or a shelf life that exceeds 60 months, such as a shelf life of 72 months; or the shelf life could be about 48 months to about 60 months, or about 55 months to about 65 months; or about 58 months to about 62 months.

Response factors relative to scopolamine and atropine were determined for the following related substances: scopolamine n-oxide, scopolamine, tropic acid, atropine, atropine n-oxide, aposcopolamine, apoatropine n-oxide, apoatropine, and atropic acid. The method was qualified using product concentrations of 3.0 mg/mL for scopolamine hydrogen bromide trihydrate and 2.0 mg/mL for atropine sulfate. The method demonstrated acceptable accuracy (100±0.5%) and precision (% RSD range 0.2 to 0.8%) for both scopolamine and atropine across the range of 50 to 150% of labeled concentration. System suitability acceptance criteria was established for scopolamine and atropine peak retention times, peak areas, tailing factors, detectability (at 0.1%) peak area precision and check standard agreement.

Scopolamine and atropine have a common hydrolysis degradation pathway that produces tropic acid from scopine from both scopolamine and atropine. Specifically, the ester bond hydrolyses to tropic acid and tropane. Tropic acid is the predominate/primary degradant product in scopolamine and atropine drug product formulations. Therefore, the tropic and atropic acids detected in stability samples via photodiode array may be from either drug substance or, more likely, both.

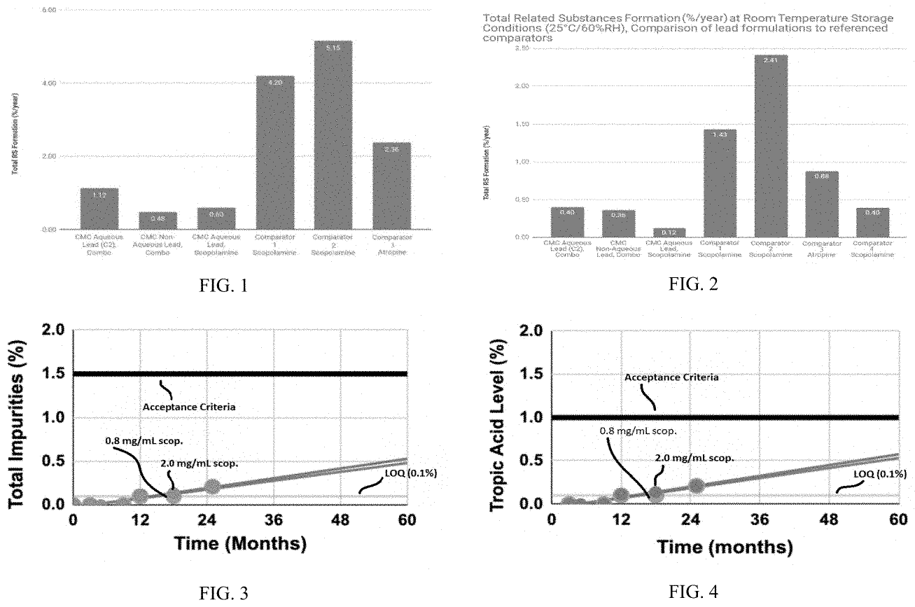

A method suitable for the assessment of scopolamine hydrobromide and atropine sulfate assay and related substances in injectable, aqueous formulations, optimized for use in formulation development studies, is presently disclosed. The method utilizes a reverse phase UPLC (ultra performance liquid chromatography) column, a gradient mobile phase, and an ultraviolet photodiode detector. The method separates and detects the main peaks of scopolamine, atropine, and primary related substances. Refer to FIG. 1 for a chromatogram of the analyte peaks and their related substances.

A method qualification was performed by assessing system suitability parameters, accuracy, precision and relative response factors compared the presently disclosed formulations with related substances commercially available. The system suitability results for three analytical runs met the preliminary system suitability criteria. These qualification results support the acceptable accuracy and precision of the method, and the conclusion that there was no matrix effect on quantitation. Relative response factors (RRF) for scopolamine and atropine related substances for which authentic materials could be obtained were also determined. This UPLC method was used to assess the stability of developed formulations.

The initial conditions of the gradient include organics at time zero, which was found to avoid stationary phase collapse and permit faster equilibration of the column compared to 100% aqueous mobile phase at the gradient start. Gradient conditions with resolved peaks of interest are presented in Table 1. The instrument conditions are shown in Table 2. Table 3 shows the expected retention times (RT) of the peaks of interest and the relative retention times (RRT) to the scopolamine and atropine peaks.

| | Mobile | Mobile | Mobile |
|---|---|---|---|
| Time | Phase | Phase | Phase |
| (min) | A (%)¹ | B (%)² | C (%)³ |
| 0 | 94 | 2 | 4 |
| 1 | 94 | 2 | 4 |
| 6 | 70 | 10 | 20 |
| 10 | 70 | 10 | 20 |
| 10.2 | 20 | 80 | 0 |
| 10.3 | 94 | 2 | 4 |
| 13 | 94 | 2 | 4 |

¹Mobile phase A: 0.1% phosphoric acid in water (v/v)
²Mobile phase B: 0.1% phosphoric acid in acetonitrile: water (90:10) (v/v/v)
³Mobile phase C: 0.1% phosphoric acid in methanol: water (90:10) (v/v/v)

TABLE 2

| | |
|---|---|
| Column | Waters BEH C18, 1.7 μm, 100 × 2.1 mm |
| Column | 50 °C |
| Temperature | |
| Flow rate | 0.55 mL/min |
| Injection volume | 1 μL |
| Detection | Photodiode array, 210 nm, |
| | 1.2 nm bandwidth |
| Autosampler | 5 ± 4° C. |
| temperature | |

| | RT | RRT to | RRT to |
|---|---|---|---|
| Peak ID | (min) | scopolamine | atropine |
| Scopolamine N-oxide | 2.814 | 0.88 | 0.61 |
| Scopolamine | 3.201 | 1.00 | 0.70 |
| Tropic acid | 4.314 | 1.35 | 0.94 |
| Atropine | 4.583 | 1.43 | 1.00 |
| Atropine N-oxide | 5.321 | 1.66 | 1.16 |
| Aposcopolamine | 5.699 | 1.78 | 1.24 |
| Apoatropine N-oxide | 6.017 | 1.88 | 1.31 |
| Apoatropine | 7.478 | 2.34 | 1.63 |
| Atropic acid | 8.107 | 2.53 | 1.77 |

A representative chromatogram showing the resolution of the compounds in Table 3 using the conditions in Tables 1 and 2 is shown in FIG. 2. The preliminary acceptance criteria for the system suitability samples were established as follows: (i) the scopolamine and atropine peak areas in the five standard injections have a % RSD NMT; (ii) the scopolamine and atropine peak retention times in the five standard injections have a % RSD NMT 2.0%; (iii) the USP tailing factors of the scopolamine and atropine peaks in the first injected standard are NMT 2.3; (iv) The scopolamine and atropine peak areas in the five detectability samples have a % RSD NMT 10%; (v) the mean scopolamine and atropine assay values of the check standards must be within 98 to 102%.

Accuracy and precision were assessed using three quality control (QC) samples prepared to represent 50, 100 and 150% of the standard concentrations of scopolamine hydrobromide 375 μg/mL and atropine sulfate 250 μg/mL. Drug substance for each analyte was used to prepare the QC samples and were not corrected for water content. The quality control samples were prepared in 1 mM citrate buffer, pH 3.0 with 150 mM sodium chloride to mimic a potential final formulation and to test for a matrix effect on quantitation. Each QC sample was analyzed three times for three different analytical runs. The target concentrations of each analyte in the controls are listed below.

Low QC: scopolamine hydrobromide 0.1875 mg/mL and atropine sulfate 0.125 mg/mL.

Medium QC: scopolamine hydrobromide 0.375 mg/mL and atropine sulfate 0.250 mg/mL.

High QC: scopolamine hydrobromide 0.5625 mg/mL and atropine sulfate 0.375 mg/mL.

No acceptance criteria were established for the quality control samples. They were analyzed to determine relative response factors (RRF) for scopolamine and atropine related substances for which authentic materials could be obtained were determined by preparing a solution containing the compounds, including scopolamine and atropine, at low concentrations. However, atropic acid was evaluated separately. The solutions were prepared using USP reference standards for scopolamine hydrobromide and atropine sulfate. The water content of each USP standard was assessed by loss on drying for scopolamine and Karl Fischer titration for atropine. The concentrations of the compounds in the solution were approximately 0.01 mg/mL (0.3% of scopolamine HBr at 3.0 mg/mL and 0.5% of atropine sulfate at 2.0 mg/mL). The solutions were injected six times and average peak areas were determined e the accuracy and precision of the method.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

Presently identified are lead and backup aqueous formulations containing atropine and scopolamine that utilize excipients found in the FDA Inactive Ingredients Database for parenteral products. The stability data show very little change when the product is stored for 3 months at the long term (25° C./60% RH) and accelerated (40° C./75% RH) conditions.

Formulations containing scopolamine (3 mg/mL) and atropine (2 mg/mL) were evaluated. A bracketing approach is utilized to identify any concentration effects of the active or inactive ingredients in the event that the dose, injected volume, or scopolamine:atropine ratio change to ensure that the formulation is appropriate and stable. The drug products were evaluated at three times higher and three times lower than the specified concentration. For example, formulations at these high and low limits (i.e., 6 mg/mL atropine and 1 mg/mL scopoline in Formulation A and 0.67 mg/mL atropine and 9 mg/mL scopolamine in Formulation B).

Solubility

Scopolamine demonstrated complete solubility (>2.5 mg/mL) in water (pH 3.7), ethanol, dimethyl sulfoxide, N,N-dimethylacetamide, and N-methyl-2-pyrrolidone. Solvents that demonstrated insufficient solvation of the drug substance included glycerol, propylene glycol, polyethylene glycol 300, cottonseed oil, soybean oil, medium chain triglycerides, and castor oil. The solubility studies were performed at a concentration of 3 mg/mL for scopolamine and 2 mg/mL atropine. The solubility was determined by visual examination of the experimental solution stored at room temperature (RT) and refrigerated for 24 hours.

Solubility studies identified water, ethanol (200 proof), dimethyl sulfoxide (DMSO), N,N-dimethylacetamide (DMA), propylene glycol (PG), gamma-Cyclodextrin and beta-cyclodextrin as solvents that are both found in the FDA inactive ingredient database for parenteral products and have sufficient solubility of atropine and scopolamine. Ethanol and propylene glycol were also determined to be compatible with the analytical method, and thus formulations in these solvents were evaluated; PEG, DMSO, DMAC, glycerol were not compatible with the analytical method.

Buffer and Excipient Compatibility

Formulations utilizing citrate, acetate or tartrate were found to be compatible with the proposed aqueous formulation approaches. To adjust osmolality to isotonic, sodium chloride was utilized successfully in the aqueous formulations.

pH Optimization

Studies at 80° C. evaluating the effects of pH on stability, determined that the pH of optimal stability was approximately pH 3 for both atropine only and combination formulations. It should be noted that the optimal pH determined at elevated temperatures (i.e., 80° C.) is typically lower than the optimal pH at pharmaceutically relevant temperatures (i.e., 25° C. and 40° C.) because the dissociation constant of water to H⁺ and OH⁻ is dependent on temperature. Prototype Formulations

Presently disclosed is a formulation containing 2 mg/mL atropine sulfate monohydrate (atropine), 3 mg/mL scopolamine HBr trihydrate (scopolamine), a buffer to control pH (citrate, acetate, or tartrate), and 150 mM sodium chloride (0.9%) to adjust osmolarity to approximately isotonic (e.g., 270 to 310 mOsm) to minimize hemolysis upon injection. In some formulations, the buffer to control pH is citrate buffer and has a pH of 3.25. The pKa values for acetate, tartrate and citrate were all within 2 units of the optimal pH. The buffer concentrations selected are the lowest concentration to produce a formulation with a limited change in pH over time. All buffer components are found in the FDA Inactive Ingredient Database for parenteral products. pH values for presently disclosed formulations varied from about 3.0 to about 3.5 and may be physiological pH (e.g., the pH normally prevailing in a human body, e.g., 7.4), or within the range of formulation pH found in FDA approved products for parenteral administration. Formulation samples were analyzed at 0, 1, 2, and 3 months at 40° C., and 3 months at 25° C. Degradants were detected using UPLC. Very low levels of degradant related substances were detected in the presently disclosed formulation. The degradants comprised primarily tropic acid and apoatropine n-oxide, but also included aposcopolamine and apoatropine. Levels of tropic acid did not exceed 0.14% at 40° C. and <LOQ at 25° C. The LOQ for the analytic method presently disclosed is 0.1%. Total amount of degradant and related substances, known and unknown, were 0.3% for all aqueous formulations.

Also disclosed are non-aqueous formulations containing solvents ethanol and/or propylene glycol. These solvents were found to not interfere with the chromatography of the assay and related substances analytical method. The ethanol containing formulation exhibited lowest levels of degradants. The total related substances for the ethanol formulation at accelerated stability conditions was less than the most stable aqueous formulation (0.12% vs. 0.23%), primarily due to the absence of an increase in tropic acid. The elimination of water from the formulations minimizes the formation of degradation products by hydrolysis, e.g., the primarily hydrolytic degradation of atropine and scopolamine to tropic acid.

Table 4, below, lists aqueous and non-aqueous combination prototype formulations. Samples were placed on stability at room temperature (25° C./60%±5% relative humidity) and accelerated conditions (40° C./75%±5% relative humidity). Samples of these formulations stored at the recommended (for 3 months) and accelerated (for 1, 2, and 3 months) were tested.

TABLE 4

| | | | Total | Sodium | | | |
|---|---|---|---|---|---|---|---|
| | Buffer/ | Target | buffer | chloride | Atropine | Scopolamine | Fill Volume |
| Drug Product | Solvent | pH⁴ | (mM) | (mM) | (mg/mL) | (mg/mL) | (mL/vial)⁵ |
| Atropine- | 1 mM | 3.0 | 1.0 | 150 | 2 | 3 | 1.25± |
| scopolamine | citrate | | | | | | |
| Atropine- | 1 mM | 3.25 | 1.0 | 150 | 2 | 3 | 1.25± |
| scopolamine | citrate | | | | | | |
| Atropine- | 1 mM | 3.5 | 1.0 | 150 | 2 | 3 | 1.25± |
| scopolamine | citrate | | | | | | |
| Atropine- | 1 mM | 3.25 | 1.0 | 150 | 2 | 3 | 1.25± |
| scopolamine | citrate | | | | | | |
| Atropine- | 1 mM | 3.25 | 1.0 | 150 | 2 | 3 | 1.25± |
| scopolamine | tartaric | | | | | | |
| | acid | | | | | | |
| Atropine- | Ethanol | — | — | — | 2 | 3 | 25 mL |
| scopolamine | (200 | | | | | | |
| | proof) | | | | | | |
| Atropine- | Propyle | — | — | — | 2 | 3 | 25 L |
| scopolamine | ne | | | | | | |
| | Glycol | | | | | | |

⁴The pH of the final formulation was adjusted appropriately with dilute HCl or dilute NaOH.
⁵Aqueous formulations were filled in 2 mL amber vials with a matched stopper and seal and non-aqueous in 25 mL headspace vials.

To further evaluate the stability profile of the combination drug product, the previously prepared samples were tested at 6, 9, 12, and 18 when stored at 25° C./60% RH and 40° C./75% RH. The assay, pH and related substances were analyzed at each stability time point. All critical attributes (appearance, pH, other specified and unspecified related substances) were monitored. All critical attributes were within the acceptance criteria.

Degradation rates for the presently disclosed formulations were compared to formulations from other suppliers (Comparators 1-4).

With reference to FIGS. 3-32, the very low levels of degradants evidence a multi-year shelf life, as shown by total impurities and tropic acid levels of the formulation when stored under various conditions, e.g., 25° C./60% RH and 40° C./75% RH. The method limit of quantitation and proposed acceptance criteria are also shown. Tropic acid is the degradant observed at the highest levels in the drug product, and is expected to be the first specified impurity outside of the acceptance criteria, and thus the degradant that most impacts the shelf life. Acceptance criteria (NMT 1% tropic acid and NMT 1.5% total impurities) were assumed, and based on the acceptance criteria for known drug products, as is known in the art. If wider acceptance criteria can be justified, a longer shelf life will be possible.

The linear lines of best fit for the data collected at 40° C. suggest that the disclosed formulations will remain within the proposed acceptance criteria for greater than 5 years. The degradation rate at 25° C. is assumed to be approximately three fold slower than at 40° C., so that a shelf life of 60 months (5 years) is possible when the product is stored at room temperature. This 60-month shelf life is also consistent with the extrapolated lines of best fit for the stability data at 25° C. that suggest the presently disclosed formulations will remain within the acceptance criteria for at least 60 months. In addition, 9-month, long-term stability data of scopolamine only aqueous formulations (citrate buffer, pH 3.0) are consistent with the stability profiles of the combination product.

The combination formulation in ethanol exhibits the lowest levels of degradants. The total related substances for the ethanol formulation at accelerated stability conditions is less than the most stable aqueous formulation (0.12% vs. 0.23% after three months). The lower levels of total related substances is primarily due to the absence of an increase in tropic acid (0.02% and 0.14% in the lead non-aqueous and aqueous formulations after three months at 40° C., respectively). This is consistent with the stabilizing strategy to eliminate water from the formulations to minimize the formation of degradation products by hydrolysis (i.e. tropic acid is formed by hydrolysis of both atropine and scopolamine). Thus, the ethanol formulation is the lead non-aqueous approach.

While injectable formulations have been approved with very high levels of ethanol, formulations in one hundred percent non-aqueous solvents may cause injection site reactions or alter pharmacokinetics. A range of formulations containing concentrations of ethanol below 100% (e.g., 30% ethanol, 70% aqueous) were evaluated to determine the minimum concentration of ethanol that provides a significant improvement in drug product stability. The ethanol based non-aqueous formulations were prepared, and 1.25 mL of the formulation was placed into 2 mL Type I amber glass vials. The vials were stoppered and sealed with an aluminum seal. A sufficient number of vials were placed at accelerated (40±2° C.175%±5% relative humidity) and room temperature conditions (25±2° C./60%±5% relative humidity). The stability of the formulations were monitored and appearance, assay and related substances, pH, and chiral purity were assessed.

### Example 2

Presently disclosed formulations and comparator formulations (see Table 5, where presently disclosed formulations are designated “CMC”) have been used to evaluate the chemical compatibility of scopolamine, atropine, and the combination with buffers and other excipients, to identify aqueous formulation effects, and to support the development of analytical methods to measure assay, related substances, and chiral purity. These comparator and legacy samples are stored at long term (25±2° C./60±5% relative humidity) and accelerated conditions (40±2° C./75±5% relative humidity) in stability chambers and utilized as needed.

TABLE 5

| | | | | Fill volume |
|---|---|---|---|---|
| Supplier | Formulation | Active (mg/mL) | Lot Number | (mL/vial) |
| Comparator | 1 mg/mL atropine; 9 mg/mL | 1 mg/mL atropine | 8137 | 1.0 |
| 3 | NaCl, pH adjusted with H₂SO₄ | | | |
| CMC | 2 mg/mL scopolamine in 1 mM | 2 mg/mL | PSC-20.01 | 1.25 |
| | citrate; 150 mM NaCl; pH 3.00 | scopolamine | | |
| Unbuffered | 2 mg/mL scopolamine | 2 mg/mL | Lot 1 | 1.0 |
| formulation | | scopolamine | | |
| Unbuffered | 2 mg/mL scopolamine | 2 mg/mL | Lot 2 | 1.0 |
| formulation | | scopolamine | | |
| CMC | 2 mg/mL atropine/3 mg/mL | 2 mg/mL atropine; | AS (1-4) | 1.0 |
| | scopolamine in 1 mM citrate | 3 mg/mL | | |
| | butter; pH 2.5-3.5 | scopolamine | | |
| CMC | 2 mg/mL atropine in 1 mM | 2 mg/mL atropine | A (1-4) | 1.0 |
| | citrate buffer; pH 2.5-3.5 | | | |
| CMC | 2 mg/mL scopolamine in | 2 mg/mL | D2 | 1.0 |
| | ethanol (N₂ overlay) | scopolamine | | |
| CMC | 2 mg/mL scopolamine in | 2 mg/mL | D1 | 1.0 |
| | ethanol (air overlay) | scopolamine | | |

At predetermined time points, samples were pulled and tested for appearance, pH (dependent on sample availability, some formulations had insufficient number of vials to measure pH at each time point), assay, related substances, and chiral purity.

Turning to the figures, FIG. 1 presents rates of total related substance formation at 40° C. of the presently disclosed formulation and selected comparator products. FIG. 2 presents rates of total related substance formation at 25° C. of presently disclosed formulations and selected comparator products.

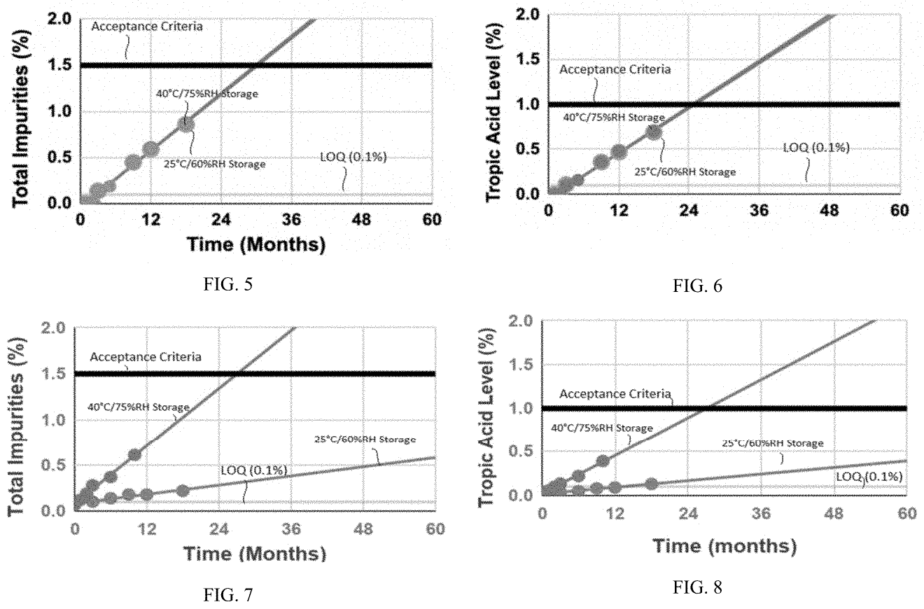

FIG. 3 shows scopolamine stability at 25° C./60% RH; 0.8 and 2.0 mg/mL, pH 3, 1 mM citrate; total impurities (%). FIG. 4 shows scopolamine stability at 25° C./60% RH; 0.8 and 2.0 mg/mL, pH 3, 1 mM citrate; tropic acid levels (%). FIG. 5 shows scopolamine stability at 40° C./75% RH; 0.8 and 2.0 mg/mL, pH 3, 1 mM citrate; total impurities (%). FIG. 6 shows scopolamine stability at 40° C./75% RH; 0.8 and 2.0 mg/mL, pH 3, 1 mM citrate; tropic acid levels (%).

Prototype formulations were prepared at bench scale (102 L), filled in 2 mL amber vials with matched elastomeric stopper and aluminum seal. As discussed above, the primary degradation pathway of both actives (scopolamine and atropine) is hydrolysis to tropic acid. Degradation of aqueous scopolamine formulations of the present disclosure was found to be approximately 2 to 10 times lower than comparator formulations 1-4. Prototypes in the analysis were formulated at 2.0 mg/mL atropine sulfate H₂O and 3.0 mg/mL scopolamine HBr.H₂O.

Regarding the aqueous formulations, controlling pH was found to strongly affect drug product stability. Aqueous formulations contained 150 mM NaCl to adjust to isotonic. Optional pH is from about 3.0 to about 3.5, changing with decreasing temperature (80° C. to 25° C.). Buffering of the present formulations is also critical to pH control, with citrate ID as lead buffer, acetate and tartrate as secondary buffers. Regarding non-aqueous solutions, elimination of water reduces formation of hydrolytic degradants (e.g., tropic acid). Solvents other than ethanol were found to not have enhanced stability.

Table 6 details formulations of the present disclosure denoted C1, C2, C3A, C4 and C5; as well as 30, 50, 70 and 100% ethanol non-aqueous formulations.

TABLE 6

| | | | | |
|---|---|---|---|---|
| | | | | Confirmed |
| | | | | Stability |
| | | | | Time |
| | | | | Point |
| Formulation | Buffer | Target pH | Ethanol | (months) |

Aqueous Formulations

| | | | | |
|---|---|---|---|---|
| C1 | 1 mM citrate | 3.00 | NA | 18 |
| C2 | 1 mM citrate | 3.25 | NA | 18 |
| C3A | 1 mM citrate | 3.50 | NA | 18 |
| C4 | 1 mM acetate | 3.25 | NA | 18 |
| C5 | 1 mM tartrate | 3.25 | NA | 18 |

Non-Aqueous Formulations

| | | | | |
|---|---|---|---|---|
| 30% EtOH | 70% 1 mM citrate | NA | 30% | 12 |
| 50% EtOH | 50% 1 mM citrate | NA | 50% | 12 |
| 70% EtOH | 30% 1 mM citrate | NA | 70% | 12 |
| 100% EtOH | NA | NA | 100% | 18 |

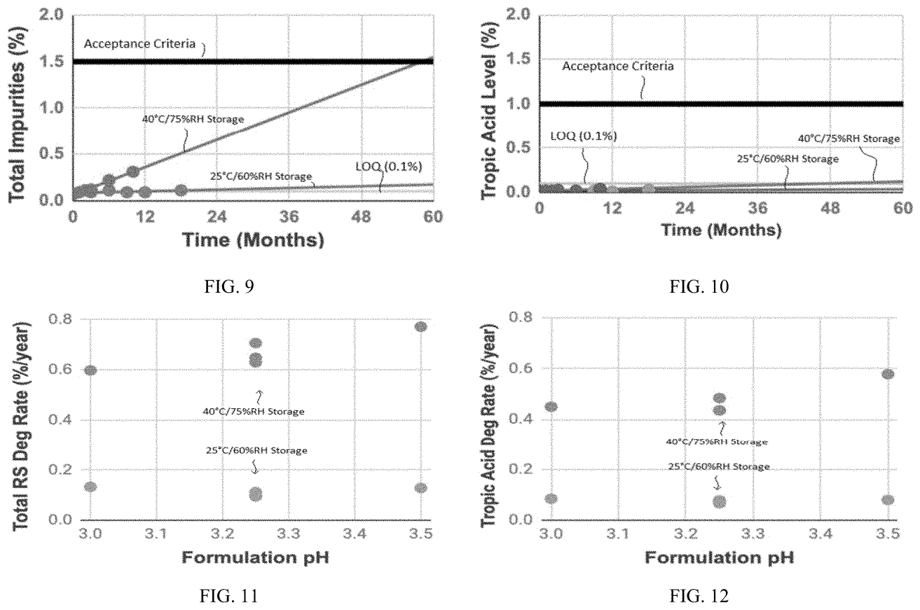

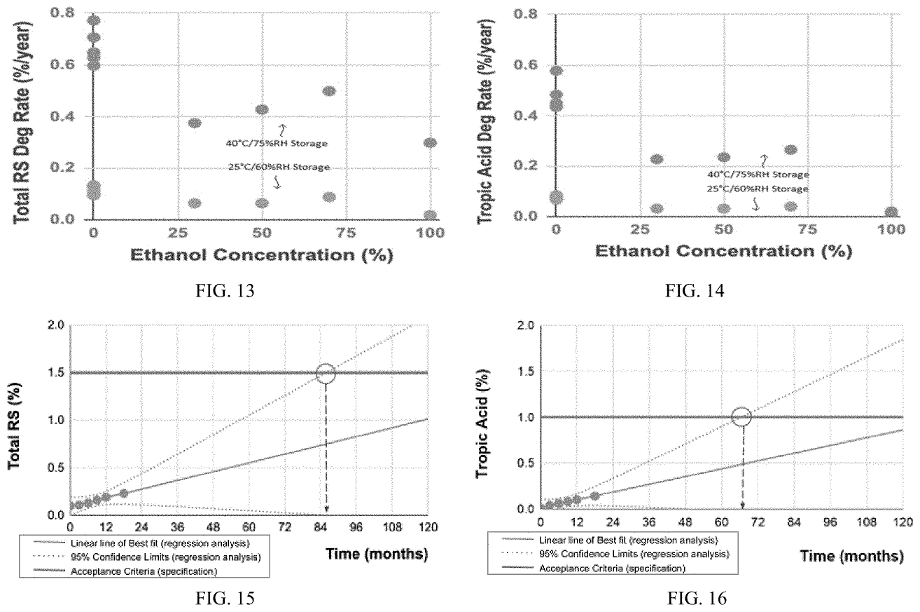

Stability times points of 12 and 18 months were taken for combo formulations were stored at 25° C./60% RH and 40° C./75% RH. Calculated degradation rates were from the slope of line of best fit. FIG. 7 shows Impurities in Formulation C2; pH 3.25, 1 mM Citrate; Total impurities (%). FIG. 8 shows Impurities in Formulation C2; pH 3.25, 1 mM Citrate; Tropic Acid levels (%). FIG. 9 shows Impurities in 100% EtOH Formulation; Total impurities (%). FIG. 10 shows Impurities in 100% EtOH Formulation; Tropic Acid levels (%). FIG. 11 shows Formulation pH Effect; Total RS Deg Rate (%/year). FIG. 12 shows Formulation pH Effect; Tropic Acid Deg Rate (%/year). FIG. 13 shows Ethanol Concentration Effect; Total RS Deg Rate (%/year). FIG. 14 shows Ethanol Concentration Effect; Tropic Acid Deg Rate (%/year). FIG. 15 shows Regression Analysis, 25° C./60% RH; Formulation=C1 Combo: 1 mM citrate, pH 3.6; Total RS (%).

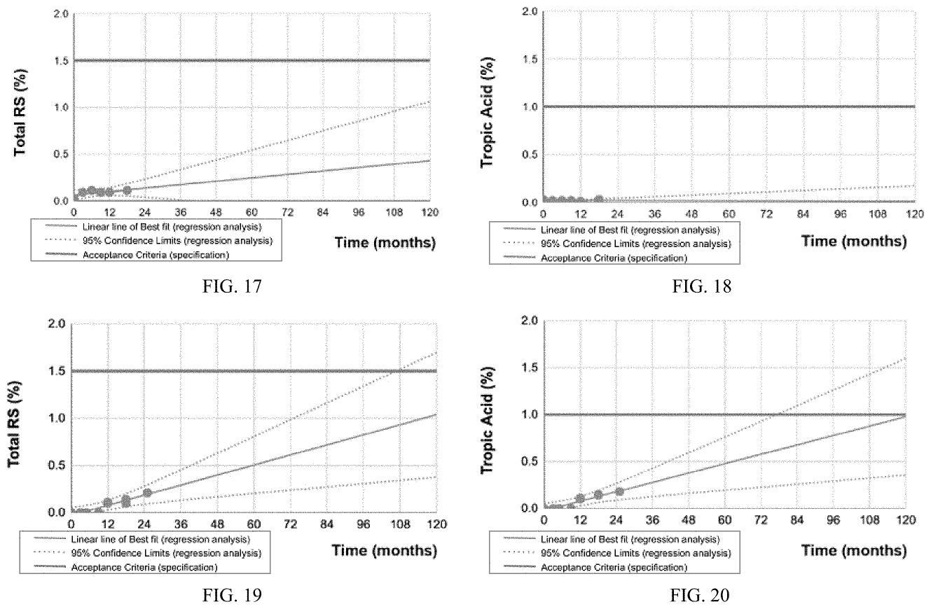

Regression analysis may be used to support long-term stability. FIG. 16 shows Regression Analysis, 25° C./60% RH; Formulation=C1 Combo: 1 mM citrate, pH 3.7; Tropic Acid (%). FIG. 17 shows Regression Analysis, 25° C./60% RH; Formulation=Combo in 100% ethanol; Total RS (%). FIG. 18 shows Regression Analysis, 25° C./60% RH; Formulation=Combo in 100% ethanol; Tropic Acid (%). FIG. 19 shows Regression Analysis, 25° C./60% RH; Formulation=Scopolamine only: 1 mM citrate, pH 3.8; Total RS (%). FIG. 20 shows Regression Analysis, 25° C./60% RH; Formulation=Scopolamine only: 1 mM citrate, pH 3.9; Tropic Acid (%).

Table 7 shows shelf-life comparisons at 25° C./60% RH.

TABLE 7

| | Maximum | | Maximum | |
|---|---|---|---|---|
| | Shelf Life | | Shelf Life | |
| | by Total RS | | by Tropic Acid | |
| | (years) | | (years) | |
| Formulation | Mean | 95% CL | Mean | 95% CL |

Aqueous Formulations

| | | | | |
|---|---|---|---|---|
| C1 | 15.3 | 7.2 | 11.6 | 5.5 |
| C2 | 11.3 | 5.2 | 12.7 | 6.0 |
| C3A | 12.8 | 6.0 | 11.6 | 5.5 |
| C4 | 14.3 | 6.7 | 11.9 | 5.7 |
| C5 | 11.8 | 5.4 | 13.8 | 6.5 |

Non-Aqueous Formulations

| | | | | |
|---|---|---|---|---|
| 30% EtOH | 22.3 | 8.7 | 30.8 | 12.0 |
| 50% EtOH | 22.3 | 8.6 | 30.8 | 12.0 |
| 70% EtOH | 16.3 | 6.6 | 24.6 | 9.5 |
| 100% EtOH | 39.3 | 14.2 | >>50 | >>50 |

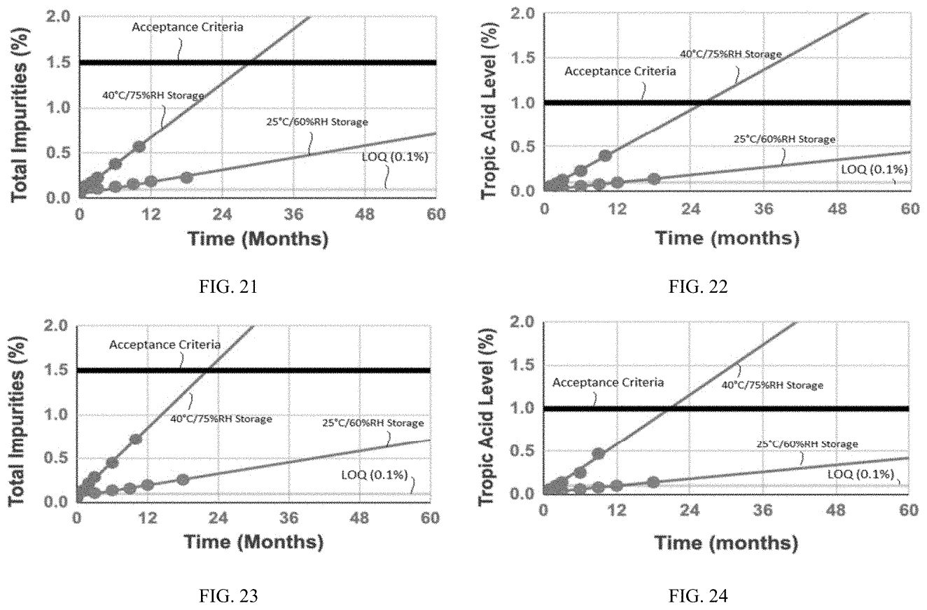

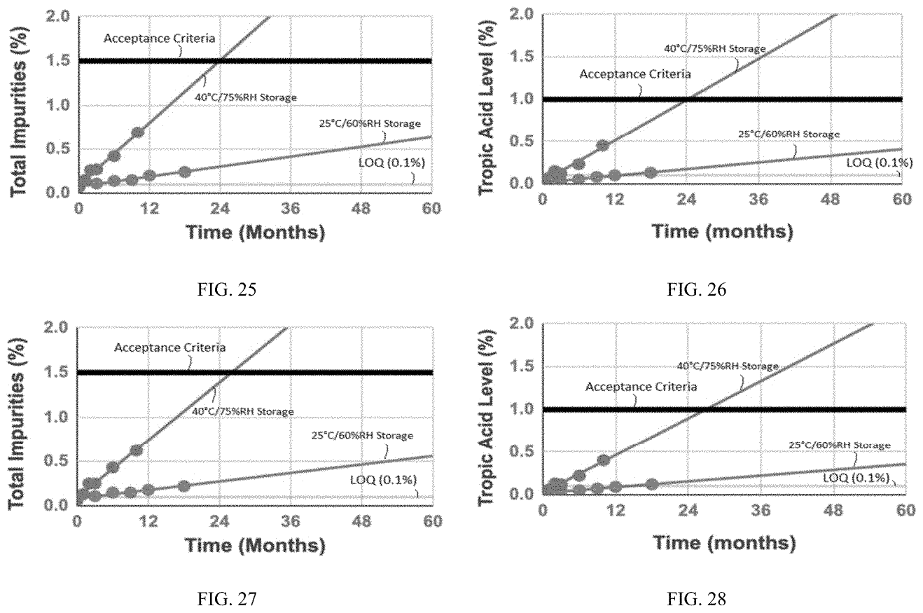

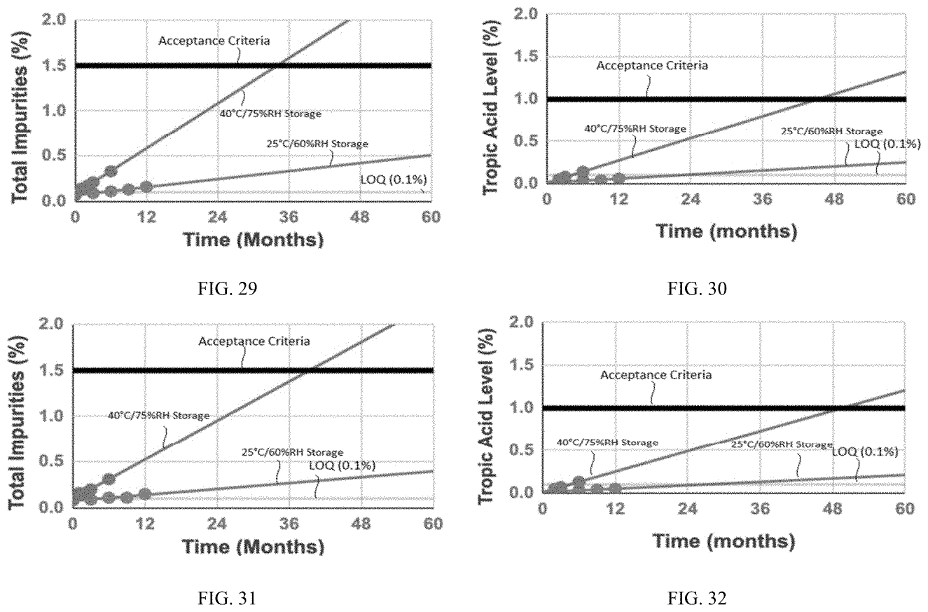

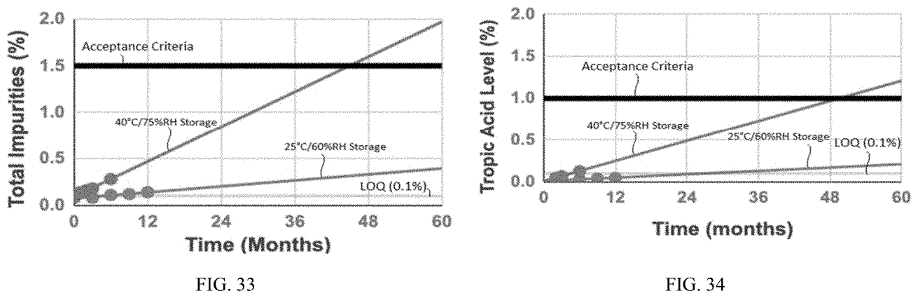

FIG. 21 shows Impurities in Formulation C1; pH 3.0, 1 mM Citrate; Total impurities (%). FIG. 22 shows Impurities in Formulation C1; pH 3.0, 1 mM Citrate; Tropic Acid levels (%). FIG. 23 shows Impurities in Formulation C3; pH 3.5, 1 mM Citrate; Total impurities (%). FIG. 24 shows Impurities in Formulation C3; pH 3.5, 1 mM Citrate; Tropic Acid levels (%). FIG. 25 shows Impurities in Formulation C4; pH 3.25, 1 mM Acetate; Total impurities (%). FIG. 26 shows Impurities in Formulation C4; pH 3.25, 1 mM Acetate; Tropic Acid levels (%). FIG. 27 shows Impurities in Formulation C5; pH 3.25, 1 mM Tartrate; Total impurities (%). FIG. 28 shows Impurities in Formulation C5; pH 3.25, 1 mM Tartrate; Tropic Acid level (%). FIG. 29 shows Impurities in 70% EtOH Formulation; Total impurities (%). FIG. 30 shows Impurities in 70% EtOH Formulation; Tropic Acid levels (%). FIG. 31 shows Impurities in 50% EtOH Formulations; Total impurities (%). FIG. 32 shows Impurities in 50% EtOH Formulations; Tropic Acid levels (%). FIG. 33 shows Impurities in 30% EtOH Formulations; Total impurities (%). FIG. 34 shows Impurities in 30% EtOH Formulations; Tropic Acid levels (%).

Therefore, the present disclosure is well adapted to attain the ends and advantages mentioned as well as those that are inherent therein. It is to be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications and sequences identified by their accession numbers mentioned in this specification are herein incorporated by reference in their entireties into the specification, to the same extent as if each individual publication, patent, or patent application or sequence identified by its accession number was specifically and individually indicated to be incorporated herein by reference. In addition, citation or identification of any reference in this application shall not be construed as an admission that such reference is available as prior art to the present invention.

The following publications are disclosed by reference herein: Newmark J. Nerve agents: pathophysiology and treatment of poisoning. Semin Neurol. 2004; 24:185-96; Ganesan K, Raza S K, Vijayaraghavan R. Chemical warfare agents. J. Pharm. Bioallied Sci. 2010; 2:166-78; 48:3-21; Iyer R, Iken B, Leon A. Developments in alternative treatments for organophosphate poisoning. Toxicol Lett. 2015; 233:200-6; Tiwari P, Dwivedi S, Singh M P, Mishra R, Chandy A. Basic and modern concepts on cholinergic receptor: A review. Asian Pac J Trop Dis. 2013; 3: 413-20; Moore D H. Long-term health effects of low dose exposure to nerve agent. J Physiol Paris. 1998; 92:325-8; 39:176-80; Munro N. Toxicity of the Organophosphate Chemical Warfare Agents GA, GB, and VX, Implications for Public Protection. ENVIRON HEALTH PERSPECT. 1994; 102:18-37; Smythies J, Golomb B. Nerve gas antidotes. J R SOC MED. 2004; 97:32; Kassa J. Review of oximes in the antidotal treatment of poisoning by organophosphorus nerve agents. J TOXICOL CLIN TOXICOL. 2002; 40:803-16; Shih T-M, Rowland T C, McDonough J H. Anticonvulsants for nerve agent-induced seizures: The influence of the therapeutic dose of atropine. J PHARMACOL EXP THER. 2007; 320:154-61. 15. Janowsky D S. Central anticholinergics to treat nerve-agent poisoning. THE LANCET. 2002; 359:265-6; Nambiar M P, Gordon R K, Rezk P E, Katos A M, Wajda N A, Moran T S, et al. Medical countermeasure against respiratory toxicity and acute lung injury following inhalation exposure to chemical warfare nerve agent VX. TOXICOL APPL PHARMACOL. 2007; 219:142-50; Che M M, Conti M, Chanda S, Boylan M, Sabnekar P, Rezk P, et al. Post-exposure treatment with nasal atropine methyl bromide protects against microinstillation inhalation exposure to sarin in guinea pigs. TOXICOL APPL PHARMACOL. 2009; 239:251-7; Worek F, Kirchner T, Szinicz L. Effect of atropine and bispyridinium oximes on respiratory and circulatory function in guinea-pigs poisoned by sarin. TOXICOLOGY. 1995; 95:123-33; Perkins M W, Pierre Z, Rezk P, Song J, Oguntayo S, Morthole V, et al. Protective effects of aerosolized scopolamine against soman-induced acute respiratory toxicity in guinea pigs. INT J TOXLCOL. 2011; 30:639-49; Muggleton N G, Bowditch A P, Crofts H S, Scott E A M, Pearce P C. Assessment of a combination of physostigmine and scopolamine as pretreatment against the behavioural effects of organophosphates in the common marmoset (Callithrix jacchus). PSYCHOPHARMACOLOGY (Berl). 2003; 166:212-20; Raveh L, Weissman B A, Cohen G, Alkalay D, Rabinovitz I, Sonego H, et al. Caramiphen and Scopolamine Prevent Soman-Induced Brain Damage and Cognitive Dysfunction. NEUROTOXICOLOGY. 2002; 23:7-17; Koplovitz I, Schulz S. Perspectives on the Use of Scopolamine as an Adjunct Treatment to Enhance Survival Following Organophosphorus Nerve Agent Poisoning. MIL MED. 2010; 175:878-82; Goodman & Gilman's: The Pharmacological Basis of Therapeutics, 13e|AccessMedicine|MCGRAW-HILL MEDICA|[Internet] [cited 2019 Mar. 21]. Available from: https://accessmedicine.mhmedical.com/book.aspx?bookid=2189; C. H. Yen, et al., Development and application of a validated UHPLC method for the determination of atropine and its major impurities in antidote treatment nerve agent auto-injectors (ATNAA) stored in the strategic national stockpiles, PHARMACOLOGY & PHARMACY, 8 (2017) 15-31.

What is claimed is:

1. A composition to combat organophosphate nerve agent threats comprising:
atropine;
scopolamine; and
ethanol;
wherein the composition comprises less than 0.14% by weight of degradants twenty-four (24) or more months post synthesis.

2. The composition of claim 1 further comprising a buffer.

3. The composition of claim 2, wherein the buffer is selected from the group consisting of citrate, acetate, and tartrate.

4. The composition of claim 1, further comprising 150 mM sodium chloride.

5. The composition of claim 1, wherein the composition has approximately isotonic osmolarity.

6. The composition of claim 5, wherein the osmolarity of the composition is from about 270 mOsm to about 310 mOsm.

7. The composition of claim 1, wherein the composition has an approximately physiological pH.

8. The composition of claim 1, wherein the composition has a pH of about 3.25.

9. The composition of claim 1, wherein the composition comprises less than 0.14% by weight of degradants sixty months post synthesis.

10. A method, comprising:
providing a subject exhibiting a cholinergic crisis subsequent to an organophosphate compound exposure;
providing a composition comprising atropine, scopolamine and ethanol that is twenty-four (24) or more months removed from synthesis; and
administering said composition to said subject under conditions such that said cholinergic crisis is reduced.

11. The method of claim 10, wherein said composition comprising atropine and scopolamine is thirty-six (36) or more months removed from synthesis.

12. The method of claim 10, wherein said composition comprising atropine and scopolamine is sixty (60) or more months removed from synthesis.

13. The method of claim 10, wherein said composition comprising atropine and scopolamine is administered less than forty (40) minutes after said organophosphate compound exposure.

14. The method of claim 10, wherein said comprising atropine and scopolamine is administered forty (40) or more minutes after said organophosphate compound exposure.

15. The method of claim 10, wherein said cholinergic crisis comprises muscular weakness, muscular paralysis, respiratory insufficiency, and pallor perspiration.

16. The method of claim 10, wherein said cholinergic crisis comprises consciousness alteration, hallucinations, seizures, respiratory center inhibition, and muscle paralysis.

17. The method of claim 10, wherein said organophosphate compound is a nerve agent.

18. The method of claim 10, wherein said organophosphate compound is a pesticide.

19. The method of claim 10, wherein said organophosphate compound is soman.

20. The method of claim 10, wherein said organophosphate compound is selected from the group consisting of tabun, sarin, cyclosarin, N,N-diethyl-2-(methyl-(2-methylpropoxy) phosphoryl)sulfanylethanamin, and O-ethyl S-[2-(diisopropylamino) ethyl] methylphosphonothioate.
